(19) United States
(12) Patent Application Publication
(10) Pub. No.: US 2011/0218339 A1
(43) Pub. Date: Sep. 8, 2011

(54) Crystalline polymorph of biopterin and production method thereof

(75) Inventors: Hideo Kawachi, Takasago-shi (JP); Yasuyoshi Ueda, Takasago-shi (JP)

(73) Assignee: KANEKA CORPORATION

(21) Appl. No.: 12/659,397

(22) Filed: Mar. 8, 2010

(51) Int. Cl. C07D 475/04

(52) U.S. Cl. 544/258

(57) ABSTRACT

Crystalline solids A to E of biopterin are distinguished from each other by diffraction angle in an X-ray powder diffraction pattern measured using Cu—Kα radiation. The crystalline solid A is characterized by strong peak at 4.6° and peaks at 13.6°, 18.1° and 27.5°; the crystalline solid B is characterized by strong peak at 4.85° and peaks at 2.4°, 13.2°, 18.1° and 27.3°; the crystalline solid C is characterized by strong peak at 5.35° and peaks at 10.8°, 21.9° and 27.3°; the crystalline solid D is characterized by strong peak at 5.1° and peaks at 2.6°, 9.2°, 13.4°, 15.4°, 18.3°, 21.8° and 27.3°; and the crystalline solid E is characterized by strong peaks at 4.5° and 5.8°, and peaks at 10.6°, 15.6°, 20.0°, 20.7°, 23.8° and 27.3°.

### BACKGROUND OF THE INVENTION

1. Field of the Invention

The present invention relates to a crystalline polymorph of biopterin and production method thereof.

2. Description of the Related Art

Biopterin is known as an intermediate for synthesizing BH4, i.e. 5,6,7,8-tetrahydro-L-erythro-biopterin. BH4 is used as a curative drug for malignant phenylketonuria and is expected to be an effective curative drug for BH4-responsive hyperphenylalaninemia and blood vessel-related diseases; therefore, biopterin, which is an intermediate for synthesizing BH4, is also extremely useful.

A number of methods for obtaining solid biopterin have been reported in the past. For example, Patent Document 1 discloses a method for precipitating biopterin by treating an alkaline aqueous solution of crude biopterin with a hydrophilic organic solvent so as to precipitate an alkaline salt of biopterin, and dissolving the precipitated alkaline salt of biopterin in water, and then adding the aqueous solution to an acidic aqueous solution. In addition, Patent Document 2 discloses a method for precipitating biopterin by adding ammonia water in order to neutralize a reaction liquid of biopterin generated by a reaction in hydrochloric acid. Moreover, Patent Document 3 discloses a method for obtaining solid biopterin by dissolving a biopterin-containing solid in dilute aqueous acetic acid (water:acetic acid=10:2) and then cooling the solution. Furthermore, Patent Document 4 discloses a method for obtaining solid biopterin by dissolving biopterin in ammonia water, concentrating the solution in vacuo and then cooling the solution; and also discloses a method for recrystallization by dissolving the obtained solid in 20% aqueous acetic acid and then cooling the solution. However, no investigations have been made into crystal polymorphs of biopterin.

Patent Document 1: JP5-37151B

Patent Document 2: WO2006-070902

Patent Document 3: JP2575781B

Patent Document 4: JP2843592B

### BRIEF SUMMARY OF INVENTION

The present inventors found novel crystalline polymorph of biopterin, and classified the crystalline polymorph into crystalline solids A to E. Preferable crystalline solids are excellent in stability, isolation property from fluid, and the like.

The crystalline solid A of biopterin is characterized by an X-ray powder diffraction pattern having strong peak at 4.6° and peaks at 13.6°, 18.1° and 27.5° in degrees 2θ±0.1, measured using Cu—Kα radiation. The crystalline solid A can be produced by adding an acid aqueous solution to an alkaline aqueous solution containing biopterin and a hydrophilic organic solvent for precipitation. The alkaline aqueous solution is preferably an aqueous solution of an alkali metal hydroxide, especially an aqueous solution of sodium hydroxide or potassium hydroxide. The hydrophilic organic solvent is preferably methanol or ethanol. The acid aqueous solution is preferably an aqueous solution of an organic acid, especially an aqueous solution of formic acid or acetic acid.

The crystalline solid B of biopterin is characterized by an X-ray powder diffraction pattern having strong peak at 4.85° and peaks at 2.4°, 13.2°, 18.1° and 27.3° in degrees 2θ±0.1, measured using Cu—Kα radiation. The crystalline solid B can be produced by adding an acid aqueous solution dropwise to a stirred alkaline aqueous solution containing biopterin, to precipitate biopterin. The alkaline aqueous solution is preferably an aqueous solution of an alkali metal hydroxide, especially an aqueous solution of sodium hydroxide or potassium hydroxide. The acid aqueous solution is preferably an aqueous solution of hydrogen chloride, sulfuric acid, formic acid or acetic acid. The crystalline solid B can be also produced by suspending biopterin in an aqueous solution of isopropanol.

The crystalline solid C of biopterin is characterized by an X-ray powder diffraction pattern having strong peak at 5.35° and peaks at 10.8°, 21.9° and 27.3° in degrees 2θ±0.1, measured using Cu—Kα radiation. The crystalline solid C of biopterin can be produced by suspending biopterin in at least one selected from methanol, ethanol and isopropanol; and can be also produced by suspending biopterin in an aqueous solution of methanol, an aqueous solution of ethanol or an mixed aqueous solution of methanol and ethanol.

The crystalline solid D of biopterin is characterized by an X-ray powder diffraction pattern having strong peak at 5.1° and peaks at 2.6°, 9.2°, 13.4°, 15.4°, 18.3°, 21.8° and 27.3° in degrees 2θ±0.1, measured using Cu—Kα radiation. The crystalline solid D can be produced by adding an alkaline aqueous solution dropwise to a stirred acid aqueous solution containing biopterin, to precipitate biopterin. The acid aqueous solution is preferably an aqueous solution of a mineral acid, especially hydrochloric acid. The alkaline aqueous solution is preferably an aqueous solution of an alkali metal hydroxide, especially an aqueous solution of sodium hydroxide or potassium hydroxide. Before adding the alkaline aqueous solution dropwise, the acid aqueous solution may be mixed with at least one alcohol. The alcohol can be exemplified by methanol, ethanol, isopropanol and the like.

The crystalline solid E of biopterin is characterized by an X-ray powder diffraction pattern having strong peaks at 4.5° and 5.8°, and peaks at 10.6°, 15.6°, 20.0°, 20.7°, 23.8° and 27.3° in degrees 2θ±0.1, measured using Cu—Kα radiation. The crystalline solid E can be produced by suspending biopterin in hexane.

In the present specification, the term “crystal polymorph” means both different crystal structures of the same substance and forms known as “pseudopolymorph” such as hydrate or solvate. In addition, crystalline solids, i.e. crystal polymorph, of biopterin are not limited to perfect crystals and may partly contain amorphous substances. When crystalline solids contain amorphous solids, the powder X-ray diffraction peaks become broad or some of the peaks can be difficult to be identified in some cases. Even if some of the powder X-ray diffraction peaks are difficult to be identified, the substance is regarded as a crystalline solid. In addition, such a crystalline solid may be described simply as a crystal.

### BRIEF DESCRIPTION OF DRAWINGS

FIG. 1 is an X-ray powder diffraction pattern data of the crystalline solid A obtained by Example 1.

FIG. 2 is an X-ray powder diffraction pattern data of the crystalline solid B obtained by Example 2.

FIG. 3 is an X-ray powder diffraction pattern data of the crystalline solid B obtained by Example 3.

FIG. 4 is an X-ray powder diffraction pattern data of the crystalline solid B obtained by Example 4.

FIG. 5 is an X-ray powder diffraction pattern data of the crystalline solid B obtained by Example 5.

FIG. 6 is an X-ray powder diffraction pattern data of the crystalline solid B obtained by Example 6.

FIG. 7 is an X-ray powder diffraction pattern data of the crystalline solid C obtained by Example 7.

FIG. 8 is an X-ray powder diffraction pattern data of the crystalline solid C obtained by Example 8.

FIG. 9 is an X-ray powder diffraction pattern data of the crystalline solid D obtained by Example 9.

FIG. 10 is an X-ray powder diffraction pattern data of the crystalline solid E obtained by Example 10.

FIG. 11 is an X-ray powder diffraction pattern data of the crystalline solid F obtained by Reference Example 1.

FIG. 12 is an X-ray powder diffraction pattern data of the crystalline solid G obtained by Reference Example 2.

FIG. 13 is a microscope photograph of the crystalline solid B obtained by Example 2.

FIG. 14 is a microscope photograph of the crystalline solid B obtained by Example 3.

FIG. 15 is a microscope photograph of the crystalline solid B obtained by Example 4.

FIG. 16 is a microscope photograph of the crystalline solid C obtained by Example 8.

FIG. 17 is a microscope photograph of the crystalline solid E obtained by Example 10.

### DETAILED DESCRIPTION OF INVENTION

1. Crystalline solid A

The crystalline solid A of biopterin is characterized by an X-ray powder diffraction pattern having strong peak at 4.6° and peaks at 13.6°, 18.1° and 27.5° in degrees 2θ±0.1, measured using Cu—Kα radiation. The crystalline solid A is stable at ambient temperature and atmosphere, and does not change into other crystalline solids even after long-term storage under general conditions, such as avoiding direct sunlight and high temperatures and humidities.

The crystalline solid A can be produced, for example, by adding an acid aqueous solution to an alkaline aqueous solution containing biopterin and a hydrophilic organic solvent for precipitation. The procedure for incorporating, especially dissolving, biopterin and the hydrophilic organic solvent in the alkaline aqueous solution is not particularly limited; but, it can be exemplified that biopterin is incorporated, especially dissolved, in the alkaline aqueous solution and then the solution is mixed with the hydrophilic organic solvent.

The alkali used in the above alkaline aqueous solution is not limited. For example, alkali metal hydroxides such as lithium hydroxide, sodium hydroxide, potassium hydroxide; alkaline-earth metal hydroxides such as barium hydroxide; alkali metal carbonates such as sodium carbonate and potassium carbonate; alkali metal hydrogen carbonates such as sodium hydrogen carbonate and potassium hydrogen carbonate; ammonia water and the like can be exemplified. The alkalis can be used singly or as a combination of two or more thereof. The alkali is preferably alkali metal hydroxides, especially sodium hydroxide, potassium hydroxide and the like.

The concentration of the alkali in the alkaline aqueous solution is, for example, about 0.01 mol/L or more and 1 mol/L or less, preferably about 0.03 mol/L or more and 0.5 mol/L or less, more preferably about 0.05 mol/L or more and 0.1 mol/L or less.

An organic solvent which can be easily mixed with water may be used as the hydrophilic organic solvent mixed (dissolved) in the above alkaline aqueous solution. As such an organic solvent, alcohols, especially C₁₋₄ alcohols, such as methanol, ethanol, isopropanol; kotones such as acetone and methyl ethyl ketone; ethers such as diethylether, tetrahydrofuran, 1,4-dioxane; nitriles such as acetonitrile; amines such as triethylamine and pyridine; amides such as dimethylformamide, dimethylacetoamide, N-methylpyrrolidone are exemplified. The hydrophilic organic solvents can be used singly or as a combination of two or more thereof. The hydrophilic organic solvent is preferably alcohols, more preferably methanol, ethanol and the like, especially preferably ethanol.

The ratio of the hydrophilic organic solvent is, for example, about 1 or more to 500 or less parts by volume, preferably about 10 or more to 200 or less parts by volume, more preferably about 30 or more to 100 or less parts by volume, relative to 100 parts by volume of water in the hydrophilic organic solvent.

The concentration of biopterin in the mixed solution of the alkaline aqueous solution and the hydrophilic organic solvent is not particularly limited insofar as it is possible to precipitate biopterin by dissolving biopterin in the mixed solution and then adding the acidic aqueous solution. The concentration is, for example, about 0.1 g/L or more and 20 g/L or less, preferably about 0.5 g/L or more and 10 g/L or less, and more preferably about 1 g/L or more and 5 g/L or less.

The acid for the above acid aqueous solution is not particularly limited. As the acid, mineral acids such as hydrochloric acid, sulfuric acid, nitric acid, phosphoric acid; and organic acids such as carboxylic acids such as formic acid and acetic acid, and sulfonic acids such as methanesulfonic acid and p-toluenesulfonic acid. The acids can be used singly or as a combination of two or more thereof. The acid is preferably organic acid, more preferably carboxylic acid, and particularly preferably acetic acid.

The concentration of the acid in the acid aqueous solution is, for example, about 0.01 mol/L or more and 1 mol/L or less, preferably about 0.03 mol/L or more and 0.5 mol/L or less, and more preferably about 0.05 mol/L or more and 0.1 mol/L or less.

The use amount of acid can be appropriately adjusted within a range whereby biopterin in the alkaline aqueous solution is precipitated through neutralization. The amount of the acid is, for example, about 0.8 or more and 1.2 or less equivalent amount, about 0.9 or more and 1.1 or less equivalent amount, and more preferably about 0.95 or more and 1.05 or less equivalent amount, relative to the alkali.

2. Crystalline solid B

The crystalline solid B of biopterin is characterized by an X-ray powder diffraction pattern having strong peak at 4.85° and peaks at 2.4°, 13.2°, 18.1° and 27.3° in degrees 2θ±0.1, measured using Cu—Kα radiation. Crystalline solid B is stable at ambient temperature and atmosphere, and does not change into other crystalline solids even after long-term storage under general conditions, such as avoiding direct sunlight and high temperatures and humidities.

2-1. First Production Method

The crystalline solid B can be produced, for example, by adding an acid aqueous solution to an alkaline aqueous solution containing biopterin, to precipitate biopterin. When the acid aqueous solution is added, it is preferable to stir the alkaline aqueous solution containing biopterin. By adding the acidic aqueous solution dropwise under stirring, it is possible to precipitate spherical particles of biopterin crystalline solid B having a uniform particle diameter. Moreover, the intensity of the stirring should be such that the precipitated crystalline solid of biopterin does not settle out.

The above-mentioned alkaline aqueous solution can be one similar to that explained in “1. Crystalline Solid A”.

The concentration of biopterin in the alkaline aqueous solution is not particularly limited insofar as it is possible to precipitate biopterin by dissolving biopterin in the alkaline aqueous solution and then adding the acidic aqueous solution. The concentration is, for example, about 0.1 g/L or more and 20 g/L or less, preferably about 1 g/L or more and 10 g/L or less, and more preferably about 3 g/L or more and 8 g/L or less.

The kind of the acid used for the acidic aqueous solution, the concentration of the acid in the acidic aqueous solution, and equivalent amount of the acid relative to the alkali are ones similar to those explained in “1. Crystalline Solid A”. However, the acid is, for example, preferably mineral acids such as hydrochloric acid and sulfuric acid, and carboxylic acids such as formic acid and acetic acid, unlike the case of crystalline solid A.

The spherical biopterin crystalline solid B, which is obtained as described above, is stable, has good filtration properties when subjected to solid-liquid separation, and has excellent powder characteristics after drying. The particle diameter of the spherical biopterin crystalline solid B can be altered by, for example, the combination of acidic aqueous solution and alkaline aqueous solution, the stirring conditions or the rate of dropwise addition; and thereby, it is possible to alter the filtration properties or the powder characteristics after drying as appropriate.

The particle diameter, i.e. mode diameter, of the spheroidized crystalline solid B is, for example, not less than 1 μm, preferably not less than 3 μm, and more preferably not less than 5 μm. The upper limit of the particle diameter, i.e. mode diameter, is not particularly limited, but the diameter can be, for example, not more than 50 μm, and particularly not more than 30 μm.

2-2. Second Production Method

The crystalline solid B of biopterin can be also produced by suspending biopterin in an aqueous solution of isopropanol. The ratio of isopropanol to water in the aqueous solution of isopropanol is arbitrary and not limited; but, the concentration of isopropanol can be, for example, 5 vol % or more and 95 vol % or less, and preferably 30 vol % or more and 95 vol % or less. According to the method, it is possible to ultimately obtain a stable biopterin crystalline solid B regardless of the earlier steps up to the point at which biopterin is suspended in the isopropanol.

3. Crystalline solid C

The crystalline solid C of biopterin is characterized by an X-ray powder diffraction pattern having strong peak at 5.35° and peaks at 10.8°, 21.9° and 27.3° in degrees 2θ±0.1, measured using Cu—Kα radiation.

The crystalline solid C is in the form of flaky crystals. The flaky crystalline solid C of biopterin can be produced, for example, by suspending other crystalline biopterin in at least one selected from methanol, ethanol and isopropanol. Alternatively, the flaky crystalline solid C of biopterin can be also produced by suspending other crystalline biopterin in an aqueous solution of methanol, an aqueous solution of ethanol and an mixed aqueous solution of methanol and ethanol. The ratio of alcohol to water in the above-mentioned aqueous solution of methanol, aqueous solution of ethanol or mixed aqueous solution of methanol and ethanol is arbitrary and not limited; but, the alcohol concentration, i.e. methanol concentration, ethanol concentration or combined methanol and ethanol concentration, is, for example, 5 vol % or more and 95 vol % or less, and preferably 30 vol % or more and 95 vol % or less. According to the production method of crystalline solid C, it is possible to easily obtain highly pure biopterin.

4. Crystalline solid D

The crystalline solid D of biopterin is characterized by an X-ray powder diffraction pattern having strong peak at 5.1° and peaks at 2.6°, 9.2°, 13.4°, 15.4°, 18.3°, 21.8° and 27.3° in degrees 2θ±0.1, measured using Cu—Kα radiation. Crystalline solid D is stable at ambient temperature and atmosphere, and does not change into other crystalline solids even after long-term storage under general conditions, such as avoiding direct sunlight and high temperatures and humidities.

The crystalline solid D can be produced by adding an alkaline aqueous solution dropwise to a stirred acid aqueous solution containing biopterin, to precipitate biopterin. Moreover, the stirring should be carried out in such a way that the precipitated crystalline solid of biopterin does not settle out.

The kind of the acid used for the acidic aqueous solution and the concentration of the acid in the acidic aqueous solution are ones similar to those explained in “1. Crystalline Solid A”. However, the acid is, for example, preferably mineral acids such as hydrochloric acid and sulfuric acid, and carboxylic acids such as formic acid and acetic acid, particularly mineral acids such as hydrochloric acid, unlike the case of crystalline solid A.

The aqueous acid solution may be mixed with at least one alcohol, before adding the alkaline aqueous solution dropwise. The procedure for dissolving biopterin and alcohol in the acidic aqueous solution is not particularly limited. For example, biopterin may be dissolved in the acidic aqueous solution and then the solution may be mixed with alcohol.

The alcohol is not particularly limited. As the alcohol, methanol, ethanol, n-propanol, isopropanol, n-butanol, isobutanol, t-butanol, n-amylalcohol, isoamylalcohol, t-amylalcohol, n-hexanol, cyclohexanol and the like can be used. The alcohols can be used singly or as a combination of two or more thereof. The alcohol is at least one selected from a group consisting of methanol, ethanol and isopropanol.

The amount of alcohol is, for example, about 1 or more and 100 or less parts by volume, preferably 5 or more and 80 or less parts by volume, more preferably 10 or more and 60 or less parts by volume, relative to 100 parts by volume of water in the acid aqueous solution.

The concentration of biopterin in the acidic aqueous solution or, the concentration of biopterin in the mixed solution of the acidic aqueous solution and alcohol if an alcohol is used, is not particularly limited insofar as it is possible to precipitate biopterin by dissolving biopterin in the solutions and adding an alkaline aqueous solution thereto. The concentration is, for example, about 0.1 g/L or more and 20 g/L or less, preferably about 0.5 g/L or more and 10 g/L or less, and more preferably about 1 g/L or more and 5 g/L or less.

The kind of the alkali used for the alkaline aqueous solution and the concentration of the alkali in the alkaline aqueous solution are ones similar to those explained in “1. Crystalline Solid A”.

The use amount of alkali can be appropriately adjusted within a range whereby biopterin in the acidic aqueous solution is precipitated through neutralization. The amount of alkali is, for example, 0.8 or more and 1.2 or less equivalent amount, preferably 0.9 or more and 1.1 or less equivalent amount, and more preferably 0.95 or more and 1.05 or less equivalent amount, relative to the acid.

5. Crystalline solid E

The crystalline solid E of biopterin is characterized by an

X-ray powder diffraction pattern having strong peaks at 4.5° and 5.8°, and peaks at 10.6°, 15.6°, 20.0°, 20.7°, 23.8° and 27.3° in degrees 2θ±0.1, measured using Cu—Kα radiation.

Crystalline solid E can be obtained by, for example, suspending a crystalline solid of biopterin in hexane, but readily changes into other crystalline solids such as crystalline solid D, and is relatively unstable. Crystalline solid E is in the form of particles, and therefore has good filtration properties when subjected to solid-liquid separation and has excellent powder characteristics after drying. Therefore, crystalline solid E is extremely useful as a form that is easy to handle in the production process.

The contents of Japanese patent application No. 2007-235563 filed on Sep. 11, 2007, and the publication thereof. No. JP2009-67695A are hereby incorporated by references.

### EXAMPLES

The present invention is more specifically described by the following examples; however, it should be understood that the scope of the present invention is not restricted by the examples described below in any way, and any appropriate modifications can be made without departing the gist of the invention described above or below, and such modifications all fall within the scope of the invention.

The purity of biopterin in the following examples and comparative examples was measured based on high performance liquid chromatography (HPLC) peak areas. The HPLC conditions were as follows.

Column: Finepak SIL C18-5 4.6 mm×250 mm

Mobile phase: methanol/phosphate buffer (pH 3)=1/4

Detector: UV detector, λ=254 nm

### Production Example 1

L-biopterin was produced under a yellow light in a one-pot process as described below.

Diethylsulfonyl-L-rhamnose (14 g, 42.1 mmol) was suspended in water (120 mL), and 4N NH₄OH was added to the stirred suspension until the pH reached 9 to 10. After the mixture was left to stand at 22° C. for 14 hours while occasionally stirred, the precipitate of diethylsulfonylmethane was filtered off, and the filtrate was dried at 40° C. under reduced pressure. The residue was dissolved in pure methanol (80 mL), and purified phenylhydrazine (5 g, 46 mmol) was added thereto. The mixture was left to stand at room temperature for 1 hour, and then dried at 40° C. under reduced pressure. The residue was washed 2 or 3 times using 50 mL of ether for each washing, dried, dissolved in pyridine (35 mL), and then cooled. Next, 35 mL of acetic anhydride ice-cooled to 0-5° C. was gradually added thereto, and then the mixture was allowed to stand in an ice bath for 10 minutes and then at room temperature for 5 hours. To the solution, methanol (200 mL) was added. The mixture was then allowed to stand at room temperature for further 10 to 15 hours, in other words, overnight.

Next, a solution obtained by dissolving sodium dithionite (1.0 g, Na₂S₂O₄) and sodium acetate.3H₂O (12.5 g) in water (300 mL) and a suspension obtained by suspending 6-hydroxy-2,4,5-triaminopyridine sulfate.H₂O in water (500 mL) were continuously added to the above methanol/pyridine solution. The whole of the obtained reaction mixture was put in a vessel in which air was substituted with nitrogen gas, sealed tightly, and then stirred for 20 hours at 35-40° C. to obtain a homogenous solution having a reddish brown color. By gradually adding a solution obtained by dissolving iodine (25 g) in methanol (300 mL) to the stirred solution, the generated tetrahydrobiopterin derivative was oxidized. The excess iodine was removed using sodium thiosulfate, and a further small quantity of iodine was added after the excess iodine was removed in order to complete the oxidation. Extremely fine brown crystals were precipitated during the oxidation process. The suspension was condensed down to approximately 100 mL, and methanol (150 mL) and 14N NH₄OH (250 mL) were added thereto. The mixture was held at 50° C. for 1 hour for deacetylation. The solution was evaporated to dryness at 40° C. under reduced pressure. The residue was suspended in methanol (100 mL) and then filtered. The obtained crude biopterin was washed with ice-cooled water (50 mL) and ethanol (200 mL), and without drying, then dissolved in boiling water (1400 mL which was minimum for dissolution). To the solution, active carbon was added. The solution was filtered while still hot, allowed to cool to room temperature, and then allowed to stand at 5° C. for 10 hours. The obtained crystals of biopterin were filtered, washed with water, washed with ethanol and ether, and then dried for 14 hours at 40° C. under reduced pressure (0.01 Torr) to obtain 6 g of L-biopterin. The yield was 60%.

### Example 1

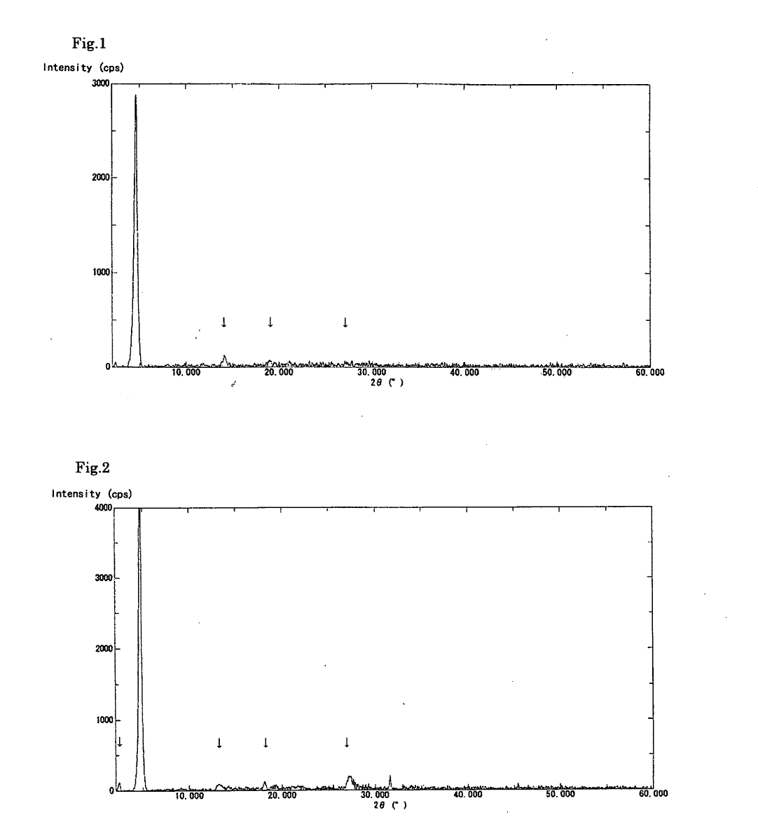

A solution was prepared by dissolving the crude biopterin (107.0 mg, 78.1% purity) obtained in Production Example 1 in a 0.1 N aqueous solution of sodium hydroxide (20 mL) and then adding ethanol (10 mL) thereto. To the stirred solution, 0.1 N aqueous acetic acid (20 mL) was added dropwise over a period of approximately 10 minutes. The precipitated crystals were separated by filtration and dried in vacuo overnight at 40° C., to obtain 81.1 mg of biopterin crystalline solid A having a purity of 91.8%. The powder X-ray diffraction data is shown in FIG. 1. In addition, the obtained crystalline solid showed no signs of changing into a different crystal form even after being stored at ambient temperature and pressure for 14 days.

### Example 2

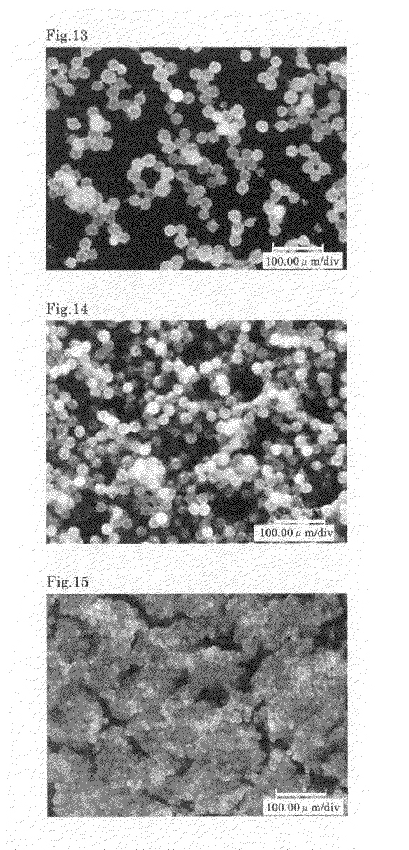

A solution was prepared by dissolving the crude biopterin (110.9 mg), which was also used in Example 1, in a 0.1 N aqueous solution of sodium hydroxide (20 mL). To the stirred solution, 0.1 N aqueous hydrochloric acid (20 mL) was added dropwise over a period of approximately 10 minutes. The precipitated crystals were separated by filtration and dried in vacuo overnight at 40° C., to obtain 83.9 mg of biopterin crystalline solid B having a purity of 91.1%. The powder X-ray diffraction data is shown in FIG. 2. The present crystalline solid exhibited superior filtration properties, compared to the crystals of Reference Example 1. In addition, the present crystalline solid showed little adhesion to the walls of the production apparatus or the packaging container. The obtained crystalline solid B was observed using a digital microscope (Keyence VHX-200); as a result, it was found that the crystalline solid B was in the form of spherical particles having a diameter of approximately 25 μm, as FIG. 13. In addition, the obtained crystalline solid showed no signs of changing into a different crystal form even after being stored at ambient temperature and pressure for 14 days.

### Example 3

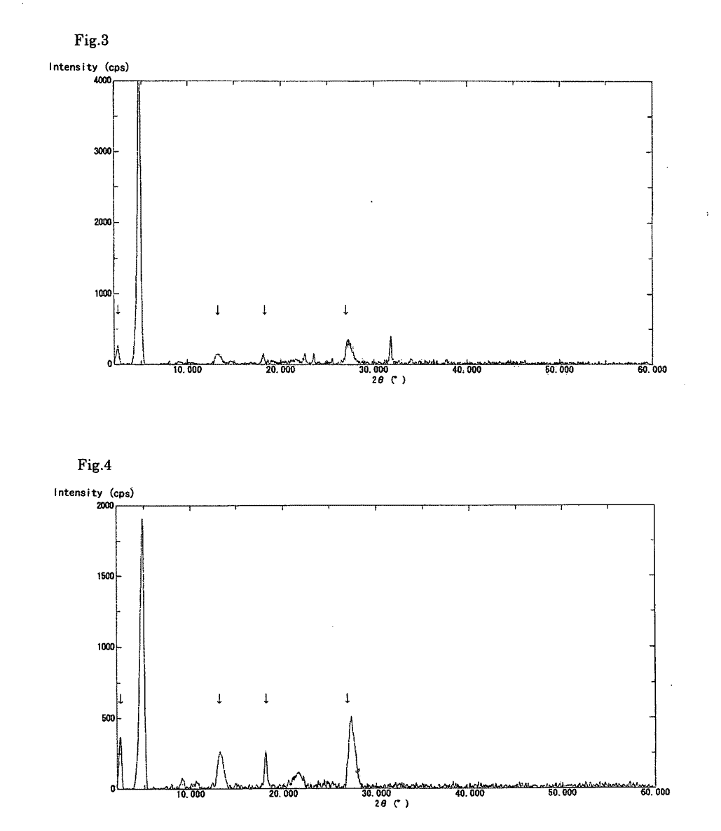

A solution was prepared by dissolving the crude biopterin (103.3 mg), which was also used in Example 1, in a 0.1 N aqueous solution of sodium hydroxide (20 mL). To the stirred solution, 0.1 N aqueous sulfuric acid (20 mL) was added dropwise over a period of approximately 10 minutes. The precipitated crystals were separated by filtration and dried in vacuo overnight at 40° C., to obtain 21.5 mg of biopterin crystalline solid B having a purity of 89.3%. The powder X-ray diffraction data is shown in FIG. 3. The present crystalline solid exhibited superior filtration properties, compared to the crystals of Reference Example 1. In addition, the present crystalline solid showed little adhesion to the walls of the production apparatus or the packaging container. The obtained crystalline solid B was observed using a digital microscope; as a result, it was found that the crystalline solid B was in the form of spherical particles having a diameter of approximately 20 μm, as FIG. 14.

### Example 4

A solution was prepared by dissolving the crude biopterin (102.1 mg), which was also used in Example 1, in a 0.1 N aqueous solution of sodium hydroxide (20 mL). To the stirred solution, 0.1 N aqueous acetic acid (20 mL) was added dropwise over a period of approximately 10 minutes. The precipitated crystals were separated by filtration and dried in vacuo overnight at 40° C., to obtain 78.9 mg of biopterin crystalline solid B having a purity of 91.1%. The powder X-ray diffraction data is shown in FIG. 4. The present crystalline solid exhibited superior filtration properties, compared to the crystals of Reference Example 1. In addition, the present crystalline solid showed little adhesion to the walls of the production apparatus or the packaging container. The obtained crystalline solid B was observed using a digital microscope; as a result, it was found that the crystalline solid B was in the form of spherical particles having a diameter of approximately 10 μm.

### Example 5

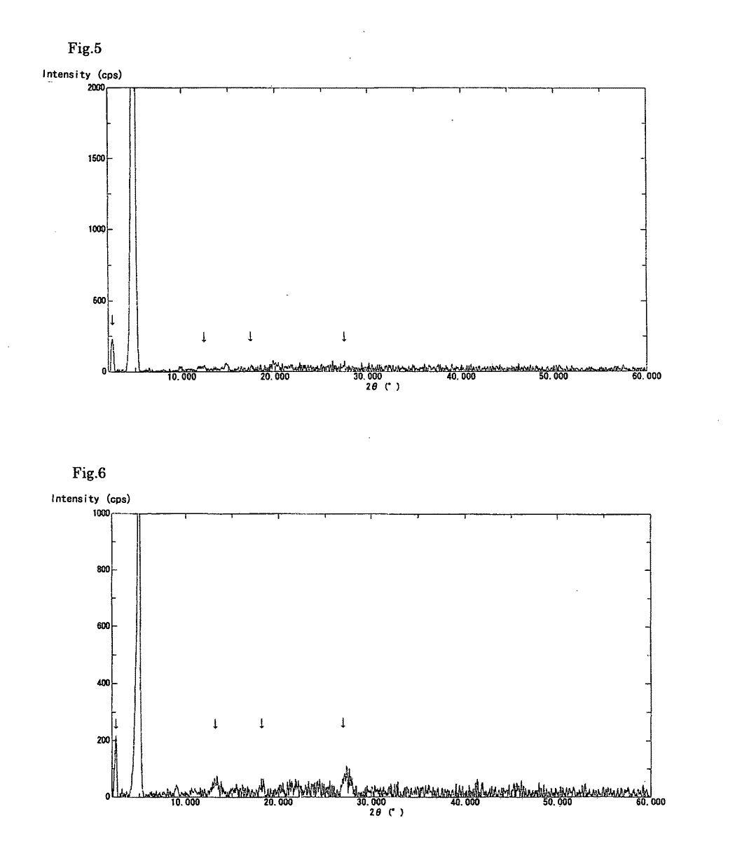

A 90% aqueous solution of isopropanol (20 mL) was added to the crude biopterin (104.5 mg), which was also used in Example 1, and the mixture was stirred for 6 hours so as to obtain a suspension. The crystals were separated by filtration and dried in vacuo overnight at 40° C., to obtain 84.0 mg of biopterin crystalline solid B having a purity of 86.5%. The present crystalline solid showed little adhesion to the walls of the production apparatus or the packaging container. The powder X-ray diffraction data is shown in FIG. 5.

### Example 6

A 50% aqueous solution of isopropanol (20 mL) was added to the crude biopterin (103.2 mg), which was also used in Example 1, and the mixture was stirred for 6 hours so as to obtain a suspension. The crystals were separated by filtration and dried in vacuo overnight at 40° C., to obtain 67.3 mg of biopterin crystalline solid B having a purity of 92.0%. The present crystalline solid showed little adhesion to the walls of the production apparatus or the packaging container. The powder X-ray diffraction data is shown in FIG. 6.

### Example 7

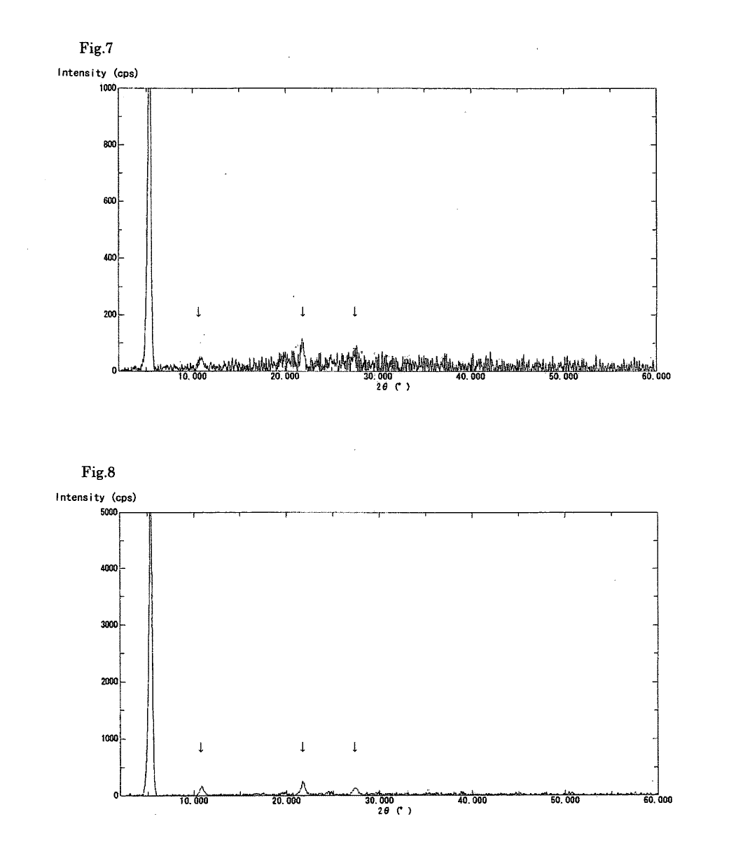

Methanol (20 mL) was added to the crude biopterin (108.8 mg), which was also used in Example 1, and the mixture was stirred for 6 hours so as to obtain a suspension. The crystals were separated by filtration and dried in vacuo overnight at 40° C., to obtain 81.8 mg of biopterin crystalline solid C having a purity of 91.4%. The powder X-ray diffraction data is shown in FIG. 7.

### Example 8

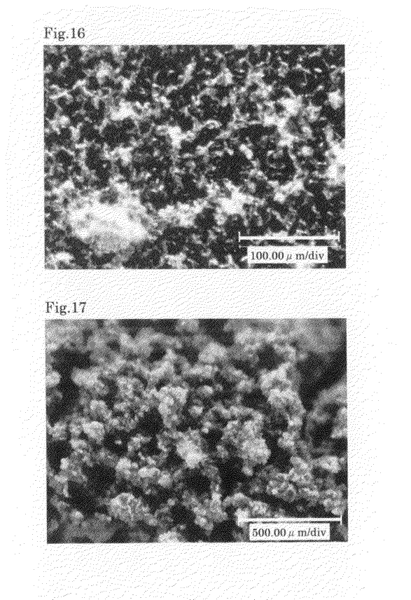

Ethanol (20 mL) was added to the crude biopterin (104.0 mg), which was also used in Example 1, and the mixture was stirred for 6 hours so as to obtain a suspension. The crystals were separated by filtration and dried in vacuo overnight at 40° C., to obtain 89.4 mg of biopterin crystalline solid C having a purity of 86.0%. The powder X-ray diffraction data is shown in FIG. 8. The obtained crystalline solid C was observed using a digital microscope; as a result, it was found that the crystalline solid C was in the form of flakes as FIG. 16.

### Example 9

The crude biopterin (101.6 mg), which was also used in Example 1, was dissolved in 0.1 N aqueous hydrochloric acid (25 mL). Isopropanol (10 mL) was added to the solution, and then a 0.1 N aqueous solution of sodium hydroxide (25 mL) was added dropwise to the stirred mixture over a period of approximately 10 minutes. The precipitated crystals were separated by filtration and dried in vacuo overnight at 40° C., to obtain 51.3 mg of biopterin crystalline solid D having a purity of 93.0%. The powder X-ray diffraction data is shown in FIG. 9. In addition, the obtained crystalline solid showed no signs of changing into a different crystal form even after being stored at ambient temperature and pressure for 14 days.

### Example 10

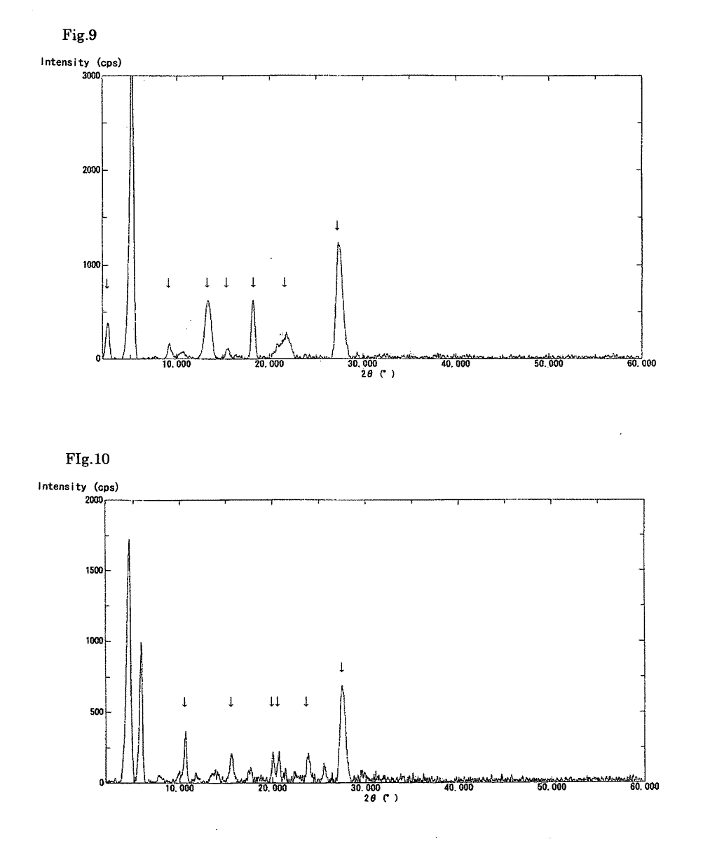

Hexane (20 mL) was added to the crude biopterin (103.9 mg), which was also used in Example 1, and the mixture was stirred for 6 hours so as to obtain a suspension. The crystals were separated by filtration and dried overnight at ambient temperature and pressure, to obtain 98.2 mg of biopterin crystalline solid E having a purity of 84.3%. The present crystalline solid was in the form of particles and had good filtering properties. The powder X-ray diffraction data is shown in FIG. 10. Moreover, the present crystalline solid was stored for 14 days at ambient temperature and pressure, and then analyzed with powder X-ray diffraction measurements again; as a result, it was confirmed that the peak pattern had changed to that of crystalline solid D.

### Reference Example 1

In accordance with the method disclosed in Japanese Publication of Examined Patent Application No. H05-37151, a crystalline solid of biopterin was obtained by dissolving a potassium salt of biopterin in water and then adding the solution dropwise to an aqueous solution of formic acid. The obtained crystalline solid of biopterin, which is called as crystalline solid F, was analyzed using powder X-ray (Cu—Kα) diffraction; as a result, a strong peak was seen at a diffraction angle (2θ±0.1) of 5.3° as FIG. 11. The strong peak was not seen in the data of the crystalline solids A to E described in the Examples, in which peaks were seen at angles of 10.5°, 15.8°, 18.1°, 19.1°, 22.0°, 22.9°, 26.7°, 28.3° and 32.0°.

### Reference Example 2

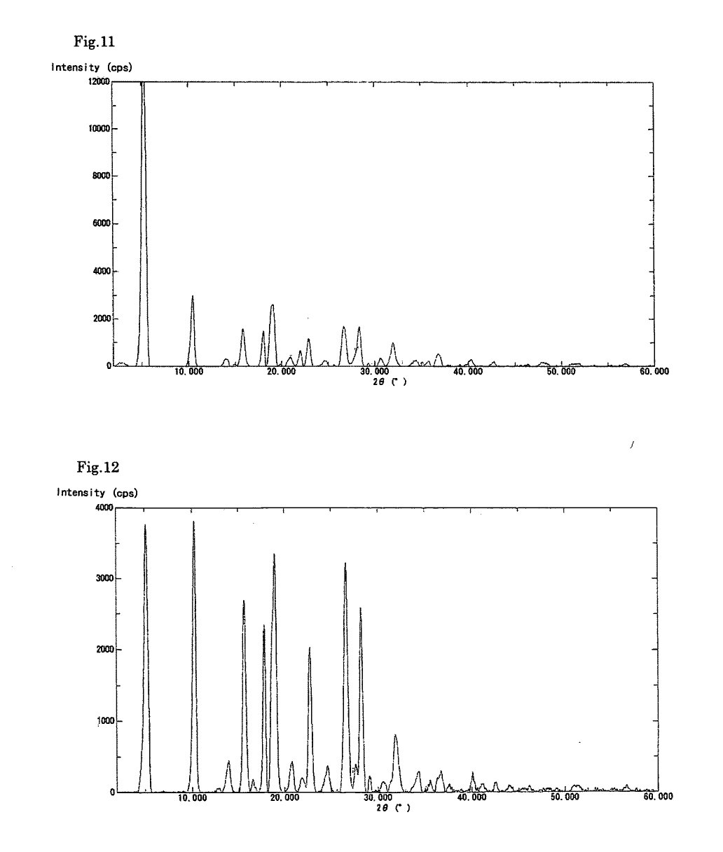

A commercially available biopterin sample (Schircks Laboratories, product no. 11.203, Lot 58), which is called as crystalline solid G, was analyzed using powder X-ray (Cu—Kα) diffraction; as a result, a strong peak was seen at a diffraction angle (2θ±0.1) of 5.1° as FIG. 12. The strong peak was not seen in the data of the crystalline solids A to E described in the Examples, in which peaks were seen at angles of 10.3°, 15.6°, 17.8°, 19.0°, 22.7°, 26.5°, 28.2° and 31.9°. The crystalline solid G was a fine powder, strongly adhered to the container, and was difficult to be discharged.

1. A crystalline solid A of biopterin, wherein the crystalline solid exhibits an X-ray powder diffraction pattern having strong peak at 4.6° and peaks at 13.6°, 18.1° and 27.5° in degrees 2θ±0.1, measured using Cu—Kα radiation.

2. A method for producing a crystalline solid A of biopterin, comprising a step of adding an acid aqueous solution to an alkaline aqueous solution containing biopterin and a hydrophilic organic solvent, to precipitate the crystalline solid A of biopterin characterized by an X-ray powder diffraction pattern having strong peak at 4.6° and peaks at 13.6°, 18.1° and 27.5° in degrees 2θ±0.1, measured using Cu—Kα radiation.

3. The method for producing a crystalline solid A of biopterin according to claim 2, wherein the alkaline aqueous solution is an aqueous solution of an alkali metal hydroxide.

4. The method for producing a crystalline solid A of biopterin according to claim 3, wherein the alkaline aqueous solution is an aqueous solution of sodium hydroxide or potassium hydroxide.

5. The method for producing a crystalline solid A of biopterin according to claim 2, wherein the hydrophilic organic solvent is methanol or ethanol.

6. The method for producing a crystalline solid A of biopterin according to claim 2, wherein the acid aqueous solution is an aqueous solution of an organic acid.

7. The method for producing a crystalline solid A of biopterin according to claim 6, wherein the acid aqueous solution is an aqueous solution of formic acid or acetic acid.

8. A crystalline solid B of biopterin, wherein the crystalline solid exhibits an X-ray powder diffraction pattern having strong peak at 4.85° and peaks at 2.4°, 13.2°, 18.1° and 27.3° in degrees 2θ±0.1, measured using Cu—Kα radiation.

9. A method for producing a crystalline solid B of biopterin, comprising a step of adding an acid aqueous solution dropwise to an alkaline aqueous solution containing biopterin with stirring, to precipitate the crystalline solid B of biopterin characterized by an X-ray powder diffraction pattern having strong peak at 4.85° and peaks at 2.4°, 13.2°, 18.1° and 27.3° in degrees 2θ±0.1, measured using Cu—Kα radiation.

10. The method for producing a crystalline solid B of biopterin according to claim 9, wherein the alkaline aqueous solution is an aqueous solution of an alkali metal hydroxide.

11. The method for producing a crystalline solid B of biopterin according to claim 10, wherein the alkaline aqueous solution is an aqueous solution of sodium hydroxide or potassium hydroxide.

12. The method for producing a crystalline solid B of biopterin according to claim 9, wherein the acid aqueous solution is an aqueous solution of hydrogen chloride, sulfuric acid, formic acid or acetic acid.

13. A method for producing a crystalline solid B of biopterin, comprising a step of suspending biopterin in an aqueous solution of isopropanol, to produce the crystalline solid B of biopterin characterized by an X-ray powder diffraction pattern having strong peak at 4.85° and peaks at 2.4°, 13.2°, 18.1° and 27.3° in degrees 2θ±0.1, measured using Cu—Kα radiation.

14. A crystalline solid C of biopterin, wherein the crystalline solid exhibits an X-ray powder diffraction pattern having strong peak at 5.35° and peaks at 10.8°, 21.9° and 27.3° in degrees 2θ±0.1, measured using Cu—Kα radiation.

15. A method for producing a crystalline solid C of biopterin, comprising a step of suspending biopterin in at least one selected from methanol, ethanol and isopropanol, or suspending biopterin in an aqueous solution of methanol, an aqueous solution of ethanol or an mixed aqueous solution of methanol and ethanol, to precipitate the crystalline solid C of biopterin characterized by an X-ray powder diffraction pattern having strong peak at 5.35° and peaks at 10.8°, 21.9° and 27.3° in degrees 2θ±0.1, measured using Cu—Kα radiation.

16. A crystalline solid D of biopterin, wherein the crystalline solid exhibits an X-ray powder diffraction pattern having strong peak at 5.1° and peaks at 2.6°, 9.2°, 13.4°, 15.4°, 18.3°, 21.8° and 27.3° in degrees 2θ±0.1, measured using Cu—Kα radiation.

17. A method for producing a crystalline solid D of biopterin, comprising a step of adding an alkaline aqueous solution dropwise to a stirred acid aqueous solution containing biopterin, to precipitate the crystalline solid D of biopterin characterized by an X-ray powder diffraction pattern having strong peak at 5.1° and peaks at 2.6°, 9.2°, 13.4°, 15.4°, 18.3°, 21.8° and 27.3° in degrees 2θ±0.1, measured using Cu—Kα radiation.

18. The method for producing a crystalline solid D of biopterin according to claim 17, wherein the acid aqueous solution is an aqueous solution of a mineral acid.

19. The method for producing a crystalline solid D of biopterin according to claim 18, wherein the acid aqueous solution is hydrochloric acid.

20. The method for producing a crystalline solid D of biopterin according to claim 17, further comprising a step of mixing the acid aqueous solution with at least one alcohol before adding the alkaline aqueous solution dropwise.

21. The method for producing a crystalline solid D of biopterin according to claim 20, wherein the alcohol is at lease one selected from methanol, ethanol and isopropanol.

22. The method for producing a crystalline solid D of biopterin according to claim 17, wherein the alkaline aqueous solution is an aqueous solution of an alkali metal hydroxide.

23. The method for producing a crystalline solid D of biopterin according to claim 22, wherein the alkaline aqueous solution is an aqueous solution of sodium hydroxide or potassium hydroxide.

24. A crystalline solid E of biopterin, wherein the crystalline solid exhibits an X-ray powder diffraction pattern having strong peaks at 4.5° and 5.8°, and peaks at 10.6°, 15.6°, 20.0°, 20.7°, 23.8° and 27.3° in degrees 2θ±0.1, measured using Cu—Kα radiation.

25. A method for producing a crystalline solid E of biopterin, comprising a step of suspending biopterin in hexane, wherein the crystalline solid E exhibits an X-ray powder diffraction pattern having strong peaks at 4.5° and 5.8°, and peaks at 10.6°, 15.6°, 20.0°, 20.7°, 23.8° and 27.3° in degrees 2θ±0.1, measured using Cu—Kα radiation.
